(19) United States
(12) Patent Application Publication
(10) Pub. No.: US 2015/0343007 A1
(43) Pub. Date: Dec. 3, 2015

(54) EDIBLE COMPOSITION COMPRISING AN EXTRACT OF INULA RACEMOSA AND NARINGIN

(71) Applicant: CONOPCO, INC. D/B/A UNILEVER, Englewood Cliffs, NJ (US)

(72) Inventors: Nandini Balaram Singh, Bangalore (IN); Gautam Banerjee, Bangalore (IN); Willemina Albertha M. Blom, Netherlands (GB); Robertus Johannes Gouka, Netherlands (GB); Ramitha Kalathil, Bangalore (IN); Suman Majumder, Kolkata (IN)

(73) Assignee: Conopco, Inc., d/b/a UNILEVER, Englewood Cliffs, NJ (US)

(21) Appl. No.: 14/649,668

(22) PCT Filed: Nov. 26, 2013
(86) PCT No.: PCT/EP2013/074735

§ 371 Date: Jun. 4, 2015

(30) Foreign Application Priority Data
Dec. 13, 2012 (EP) 12196860.6

(51) Int. Cl. A61K 36/752; A61K 31/7048; A23L 1/30; A23F 3/00; A23L 1/00; A23L 1/29; A61K 36/28; A23L 1/39

(52) U.S. Cl. CPC A61K 36/752; A61K 36/28; A61K 31/7048; A23L 1/39; A23F 3/00; A23L 1/0035; A23L 1/293; A23L 1/3002; A23V 2002/00

(57) ABSTRACT

The present invention provides relates to an edible composition for anti-diabetic benefit. Diabetes is one of the major and commonly occurring health problems in today's world. Pharmaceuticals companies are very active in this field to develop new medicines for preventing and controlling diabetes. There are several medicines available in the market for the treatment of type 2 diabetes. There are prior arts which describes composition and/or therapies for the prevention of glucose intolerance and/or diabetes. We have found that, though prior art discloses compositions and therapies for treating diabetes, there is not disclose any food composition which is effectively controls GLP-1 activity thereby control diabetes. The present inventors while working extensively for providing an edible composition for preventing diabetes have surprisingly found that a particular combination of Inula racemosa and naringin is effective for controlling and/or preventive diabetes thereby satisfying one or more of the aforesaid objects.

### FIELD OF THE INVENTION

The present invention relates to an edible composition. Most particularly the present invention relates to an edible composition for anti-diabetic benefit.

### BACKGROUND OF THE INVENTION

Diabetes is one of the major and commonly occurring health problems in today's world. Some people inherited diabetes from their parents (type 1) and some of them acquired it because of their unhealthy life style and metabolic disorder (type 2). Whether it is type 1 or type 2, in the long term diabetes can be proved to be a life threatening disease in absence of any early actions to prevent it.

Type 2 diabetes increases the level of dipeptidyl peptidase-4 (DPP-4) thereby decreasing the activity of glucagon-like peptide-1 (GLP-1). One of the major roles of GLP-1 is to maintain the blood glucose level. The reduced activity of GLP-1 because of increased DPP-4 induces the imbalance and thereby increases the blood sugar level. DPP-4 inhibits the action of GLP-1 and increases blood sugar level (Hoist et. al. 1998; Inhibition of the activity of dipeptidyl-peptidase IV as a treatment for type 2 diabetes; Diabetes, Vol. 47, 1663-1670 AND Karagiannis et. al. 2012; Dipeptidyl peptidase-4 inhibitors for treatment of type 2 diabetes mellitus in the clinical setting: systematic review and meta-analysis. British Medical Journal 344).

Pharmaceuticals companies are very active in this field to develop new medicines for preventing and controlling diabetes. There are several medicines available in the market for treating diabetes.

One of the problem with diabetes is that people generally do not consider diabetes as a serious disease as because there are no immediate visual effect of this disease. Therefore most of the people do not care take any action for preventing it. Another problem in general with medicine is that people take medicine only when they are not well. Taking medicine everyday generally has some negative psychological effect.

There are prior arts which describes composition and/or therapies for the prevention of glucose intolerance and/or diabetes.

US2012094942 (Elcelyx Therapautics Inc., 2012) discloses methods of modulating hormone concentrations in a subject comprising the administration of a composition comprising a chemosensory receptor ligand, wherein the composition is adapted to deliver the ligand to one or more regions of the intestine of said subject. Also provided are methods directed to the modulation of circulating concentrations of one or more of GLP-1 (total), GLP-1 (active), GLP-2, oxyntomodulin, PYY (total), PYY 3-36, CCK, GIP, insulin, C-peptide, glycentin, uroguanylin amylin, and ghrelin (total), ghrelin (active) and glucagon.

WO 2008/118011 (Phyto Health Pharma B.V., 2008) describes a composition for the treatment of diabetes mellitus. Even more specifically, the invention relates to a herbal composition for treating the same. In one of its embodiments, the invention provides a composition comprising a part or extract from Curcuma longa, Gymneme sylvestre, Momordica charantia, Trigonella foenum graecum and at least one Terminalia species.

US2011/274680 (Mazed, Mohammad A, et al, 2011) discloses a synergistic chemical composition of bioactive compounds in a dietary supplement for lowering the risks of Alzheimer's, Cardiovascular and Diabetes diseases. It also discloses chemical compositions of a sugar free sweetener/super sweetener for people with Type-2 Diabetes disease and nano encapsulation and targeted nano delivery of bioactive compounds and/or bioactive molecules for lowering the risks of Alzheimer's, Cardiovascular and Diabetes diseases. There is also a microelectro-mechanical system (MEMS) based passive and active delivery of bioactive compounds and/or bioactive molecules. Inula racemosa is mentioned as one of the botanicals for lowering the risk of cardiovascular disease and does not disclose a synergistic edible composition for effectively controlling GLP-1 activity.

We have found that, though prior art discloses compositions and therapies for treating diabetes, there is no disclosure of any food composition which is effectively controls GLP-1 activity thereby control diabetes.

Therefore there is a need to provide an edible food composition with anti-diabetic benefit which allows controlling GLP-1 activity without taking any medicine and in turn without any negative psychological effect.

### Objects of the Invention

It is therefore an object of the present invention to provide an edible composition for preventing diabetes.

It is another object of the present invention to provide an edible composition to control GLP-1 activity by inhibiting DPP-4.

It is yet another object of the present invention to provide for a suitable alternative for controlling and preventing diabetes.

The present inventors while working extensively for providing an edible composition for preventing diabetes have surprisingly found that a particular combination of extract of Inula racemosa and naringin is effective for controlling and/or preventive diabetes thereby satisfying one or more of the aforesaid objects.

### SUMMARY OF THE INVENTION

According to a first aspect of the present invention there is provided an edible composition comprising:
a. 0.01 to 10% by weight of extract of Inula racemosa; and
b. 0.01 to 10% by weight of naringin.

According to the second aspect of the present invention there is provided a process of producing an edible composition comprising the steps of mixing and/or blending 0.01 to 10% by weight of extract of Inula racemosa and 0.01 to 10% by weight of naringin with the other ingredients to obtain the edible composition.

Any feature of one aspect of the present invention may be utilized in any other aspect of the invention. The word “comprising” is intended to mean “including” but not necessarily “consisting of” or “composed of.” In other words, the listed steps or options need not be exhaustive. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

### DETAILED DESCRIPTION OF THE INVENTION

The present invention provides an edible composition comprising:
a. 0.01 to 10% by weight of extract of Inula racemosa; and
b. 0.01 to 10% by weight of naringin.

The term edible composition preferably means a composition which is ingestible by human being.

The edible composition preferably comprises 0.1 to 10%, more preferably 1 to 10%, further more preferably 3 to 10% and most preferably 5 to 10% by weight of extract of Inula racemosa.

Inula racemosa is a species of an ornamental plant of the Asteraceae family. Inula racemosa grows in the temperate and alpine western Himalayas, and it is common in Kashmir, and also known as “Pushkarmool”. “Extract of Inula racemosa” herein is to be understood as a composition obtainable by extracting roots of such plants or preferably parts of such roots with liquid and preferably water Herein, “extract of Inula racemosa” is the same as “Inula racemosa extract”. All the above mentioned percentage is on solid weight basis of the composition. If the composition is having high percentage of water then the percentage of the extract of Inula racemosa as mentioned above has to construe accordingly.

The edible composition also preferably comprises 0.1 to 10%, more preferably 1 to 10%, further more preferably 3 to 10% and most preferably 5 to 10% by weight of naringin.

“Naringin” herein relates to the molecular structure as set out below, and is chemically known as 4,5,7-Trihydroxyflavanone 7-rhamnoglucoside (Chemical formula: C₂₇H₃₂O₁₄, Mw=580.54), including the edible salts thereof.

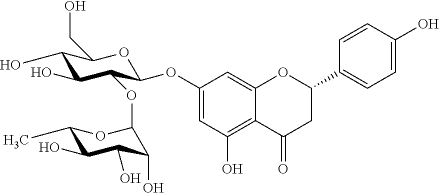

Naringin is a flavanone glycoside naturally occurs in fruits e.g. citrus fruits especially in grapes. It is one of the major flavonoid in grapefruit. The preferred source of naringin for the purpose of the present invention is from citrus fruits mainly grapes.

Preferably in the composition of the present invention, the ratio of Inula racemosa to naringin is in the range of 1:0.01 to 1:10, more preferably in the range of 1:0.1 to 1:10 and further more preferably in the range of 1:1 to 1:10 and most preferably in the range of 1:1 to 1:5.

Though the edible composition of the present invention is not limited to any particular edible composition but the preferred composition of the present invention is in the form of a liquid such as a soup or a beverage, a spread, a dressing, a dessert or bread.

The most preferred beverage is tea based beverage.

The term tea based beverage as herein referred to preferably include black tea based beverages, green tea based beverage and oolong tea based beverages. The preferable format may be liquid tea drink, ready-to-drink tea, tea juice etc. both hot and/or cold brew.

The edible composition of the present invention may also be in the form of a solid or powdered food supplement.

The present invention also provides a process of producing an edible composition comprising the steps of mixing and/or blending 0.1 to 10% by weight of extract of Inula racemosa and 0.01 to 10% by weight of naringin with the other ingredients to obtain the edible composition.

The term “other ingredients” as mentioned above means the compositional ingredients needed for making a targeted edible product e.g. in case of making a soup composition (targeted edible product) the term “other ingredients” preferably are starch, salt, sugar, yeast extract, fat powder, vegetable pieces, flavour, colour etc.

To make the edible composition of the present invention, the Inula racemosa extract may be prepared by extracting (boiling) the roots of Inula racemosa with water at a temperature in the range of 70 to 100° C. for 2-6 hours followed by cooling. After that the solution is filtered and concentrated. The concentration stage preferably carried out in a rotary evaporator.

Alternately, commercially available (if available) Inula racemosa water extract powder may also be used.

The composition of the present invention has been primarily developed for preventing and controlling diabetes and more particularly type 2 diabetes.

Without wishing to be bound by theory it is stated that type-2 diabetes associated with increasing DPP-4 (dipeptidyl peptidase-4) and thereby decreasing GLP-1 (glucagon-like peptide-1) activity. GLP-1 generally maintains the balance thereby controls blood sugar level. Increasing level of DPP-4 suppress the activity of GLP-1. The present invention is primarily developed to inhibit DPP-4 and thereby maintaining the activity of GLP-1 which in turns controls the blood sugar level.

The present invention provides the use of a composition for anti-diabetic benefit.

The present invention provides the use of a composition for the treatment of type 2 diabetes.

The present invention provides the use of a composition according for maintaining GLP-1 activity.

The present invention provides the use of a composition for the inhibition of DPP-4.

The invention will now be demonstrated with the help of examples, which are for the purpose of illustration, and in no way limit the scope of the invention.

### EXAMPLES

### Preparation of Extract of Inula racemosa

Inula racemosa extract was prepared by using the following procedure:

The Inula racemosa (pushkarmool) plant was bought from the local (Bangalore, India) market. This was available as a stem size of ˜3-6 cm which was a combination of roots and stems of pushkarmool. The dried Inula racemosa powder was then prepared by a pulverizer (cutting mill, Retsch SM 100) attached with a 200 μm size sieve.

The extract of the Inula racemosa was prepared from dried Inula racemosa powder. 100 g of dry Inula racemosa root powder was soaked in 800 mL of water for ˜14 hours and then boiled at 80° C. for 4 hours. It was then cooled down to ˜35° C. followed by filtering the solution to get a clear solution. The solution was then concentrated to dryness (moisture content of ˜3%) using rotary evaporator (Heidolph Laborota 4002). This extract was used for the other experiments as described below.

Invitro DPP-4 Enzyme Assay:

A 96 well plate (NEST Biotechnology Co. Ltd, Cat No 701001) was taken. In each well of 96 well plate extract of Inula racemosa (as prepared above), Naringin (SIGMA, Cat. No: N1376) and the combination of extract of Inula racemosa and naringin were put in varied concentration as per Table 1. In each well, 4 ng of human recombinant Dipeptidyl peptidase-IV (DPP-IV) enzyme (PROSPEC ISRAEL, Cat. No. ENZ 375) was also added. After that, the final volume was made up to 200 μl/well using TRIS-HCl buffer of pH-8. The TRIS-HCl buffer was prepared by adding 2.42 g of TRIS (Tris hydroxyl methyl amino methane; Supplier: Sisco Research Laboratory ltd, Cat No 2044122) base, 0.372 g of EDTA (SIGMA, Cat No E6758) and 5.644 g of NaCl in 900 mL of autoclaved milli-Q water (Millipore® India) and stirred it till it get dissolved (˜30 minutes). After that the pH of the solution was adjusted to 8.0 using HCl acid and then the volume of the solution was made up to 1000 mL with autoclaved milli-Q water.

The enzyme and combinations of extract of Inula racemosa and naringin were mixed for 1 min using microplate reader (BIO-RAD LAB INDIA, Model No. 680) and incubated in an incubator (Thermo Scientific, Model 3111; conditions: at 37° C.) for 10 minutes. After that the enzymatic reaction was initiated by adding 10 μL/well of the 19 mM of substrate Glycine-Proline para nitroanilide (Gly-Pro p-NA) (this is equivalent to GLP-1). Gly-Pro-p NA is a universally accepted and commercially available substrate for the enzyme DPP-4, due to the lack of availability of commercially available chromogenic GLP-1. We have used Gly-Pro-p NA in the assay which is equivalent to GLP-1. It has also been reported to use Gly-Pro-p NA instead of GLP-1 (Yogisha et. al., Journal of Natural Products, Vol. 3(2010):76-79).

As controls three different sets of samples were also taken for this assay viz. only substrate (Gly-Pro-p NA) at the same concentration used above (Control 1), only DPP-4 enzymes at the same concentration used above (Control 2) and other was the combination of Gly-Pro-p NA and DPP-4 at the same respective concentration used above (Control 3).

All the above reaction mixture was again incubated for 1 hr in the same incubator under the same condition. Then the enzymatic activity was arrested by adding 100 μL/well of citrate buffer. The citrate buffer was prepared adding 2.1 g of citric acid monohydrate (Sisco Research laboratory ltd, Cat No 0348216) and 2.94 g of Sodium citrate tri basic dihydrate (Sisco Research laboratory ltd, Cat No 1949110) in 90 mL of autoclaved milli-Q water (Millipore® India) and stirred it till it get dissolved (˜30 minutes). After that the pH of the solution was adjusted to 4.0 using HCl acid and then the volume of the solution was made up to 100 mL with autoclaved milli-Q water. After this the absorbance was measured at a wavelength of 405 nM using microplate reader (BIO-RAD LAB INDIA, Model No. 680).

To get the % inhibition by Inula racemosa and naringin, the absorbance value for the Control 3 was considered as 100% activity of DPP-4.

The % activities of the DPP-4 for the other samples were calculated using the following formula:

$$[ \frac{OD of the wells containing samples}{OD of the well containing Control 3} ] \times 100 = \% activity$$

The % inhibitions by the samples were then calculated by subtracting the activity from 100. The results of the experiments summarized below in Table 1.

TABLE 1

| | | | Ratio of | % | |
|---|---|---|---|---|---|
| Example | Inula racemosa | Naringin | Inula racemosa: | Inhi- | |
| No. | (μg/well) | (μg/well) | Naringin | bition | SD* |
| A | 100 | 0 | — | 5.61 | 0.02 |
| B | 0 | 116 | — | 18.54 | 1.42 |
| C | 0 | 232 | — | 18.43 | 5.02 |
| D | 0 | 580 | — | 18.11 | 0.70 |
| 1 | 100 | 116 | 1:1.16 | 41.02 | 7.01 |
| 2 | 100 | 232 | 1:2.32 | 42.79 | 5.11 |
| 3 | 100 | 580 | 1:5.8 | 28.93 | 0.39 |

*SD in Table 1 represents standard deviation of the population of a particular sample.

From the above table it is evident that a combination of Inula racemosa and naringin in the ratio inside the scope of the present invention (Example 1, 2 and 3) provides much higher “% inhibition” than either of Inula racemosa or naringin when used alone at the same concentration (Example A, B, C and D).

Therefore it is clear that the combination of combination of Inula racemosa and naringin provides synergistic benefit when used in particular ratios.

Preparation of Edible Compositions:

Soup Composition:

The soup composition was made by mixing the dry ingredient according to the following Table:

TABLE 2

| Ingredient | Wt % |
|---|---|
| Corn Starch | 46 |
| NaCl | 10 |
| Sugar (commonly available sucrose) | 12 |
| Yeast Extract | 2 |
| Fat Powder (Lecithin) | 4 |
| Liquid Fat (Lecithin) | 2 |
| Dried vegetable pieces | 17 |
| Flavour | 2.5 |
| Colour | 0.5 |
| Inula racemosa extract | 2 |
| Naringin | 2 |

The soup was then prepared using 15 g of the above composition in 100 mL of hot water (˜90° C.) and tasted by a group of professional taster. It was found that the addition of Inula racemosa extract and naringin did not alter the taste of the soup. The soup was as delicious as a control soup (without the addition of Inula racemosa aqueous extract and naringin).

1. An edible composition comprising:
a. 0.01 to 10% by weight of extract of Inula racemosa; and
b. 0.01 to 10% by weight of naringin.

2. A composition as claimed in claim 1 wherein the ratio of Inula racemosa to naringin is in the range of 1:0.01 to 1:10.

3. A composition as claimed in any one of the preceding claim 1 or 2 wherein the Inula racemosa extract is an extract of Inula racemosa root or parts thereof.

4. A composition as claimed in claim 3 wherein the Inula racemosa extract is a water extract.

5. A composition as claimed in any one of the preceding claims wherein the source of naringin is a fruit.

6. A composition as claimed in claim 4 wherein the fruit is a citrus fruit.

7. A composition as claimed in any one of the preceding claims in the form of a liquid such as a soup or a beverage, a spread, a dressing, a dessert or bread.

8. A composition as claimed is claim 7 wherein the beverage is a tea based beverage.

9. A composition as claimed in any one of claims in the form of a solid or powdered food supplement.

10. A process of producing an edible composition comprising the steps of mixing and/or blending 0.01 to 10% by weight of extract of Inula racemosa and 0.01 to 10% by weight of naringin with the other ingredients to obtain the edible composition.

11. A process as claimed in claim 10 wherein the ratio of Inula racemosa to naringin is in the range of 1:0.01 to 1:10.

12. A composition as claimed in any one of the preceding claims 1 to 9 for use in treating diabetes.

13. A composition as claimed in any one of the preceding claims 1 to 9 for use in treating type 2 diabetes.

14. A composition as claimed in any one of the preceding claims 1 to 9 for use in maintaining GLP-1 activity.

15. A composition as claimed in any one of the preceding claims 1 to 9 for use in the inhibition of DPP-4.
